(19) United States
(12) Patent Application Publication
(10) Pub. No.: US 2018/0339116 A1
(43) Pub. Date: Nov. 29, 2018

(54) Pouch for Cooling an Insulin Pump and a Combination of Such a Pouch, a Protective Casing and an Insulin Pump

(71) Applicant: Ivo Henry Van der Vleuten, Waspik (NL)

(72) Inventor: Ivo Henry Van der Vleuten, Waspik (NL)

(21) Appl. No.: 15/776,929

(22) PCT Filed: Nov. 21, 2016
(86) PCT No.: PCT/NL2016/050816

§ 371 Date: May 17, 2018

(30) Foreign Application Priority Data
Nov. 19, 2015 (NL) 2015817

(51) Int. Cl. A61M 5/44

(52) U.S. Cl. CPC A61M 5/44; A61M 5/14244

(57) ABSTRACT

The invention relates to a pouch for cooling an insulin pump, which pouch includes a bottom wall and a standing peripheral wall along the bottom wall for enclosing an insulin pump. The upper edge of the peripheral wall forms an insertion opening for inserting the insulin pump. At least two opposite wall parts of the peripheral wall are provided with hydrogel-filled compartments, and the wall parts are water-permeable for the purpose of bringing the hydrogel into contact with water.

The invention relates to a pouch for cooling an insulin pump, preferably 24 hours a day, which pouch comprises:
a bottom wall; and
a standing peripheral wall along the bottom wall for enclosing an insulin pump and wherein the upper edge of the peripheral wall forms an insertion opening for inserting the insulin pump.

Such a pouch is known from EP 0934050.

EP 0934050 describes a cooling pouch particularly for insulin syringes, whereby it becomes possible to keep insulin syringes outside a refrigerator or the like for a longer period of time, for instance during travel.

The cooling pouch consists of a double textile layer stitched together along the periphery and wherein stitching is arranged in longitudinal direction parallel to the edges in order to form compartments in the double textile layer. The compartments are filled with a water-absorbent granular material, such as a hydrogel, which can on the one hand absorb water and can on the other relinquish the water through evaporation.

By immersing the cooling pouch in water the hydrogel will be saturated with water. Once the textile layers have been removed and left to drain, the insulin syringes for instance can be wrapped in the pouch.

The water in the hydrogel will evaporate at the surface of the gel and thereby have a cooling effect so that the syringes remain cooled.

EP 0934050 also describes a pouch with a bottom provided with compartments filled with hydrogel. Arranged along the edge of the bottom is an upstanding wall which consists of a single-walled layer having on an upper edge a cover which is likewise provided with compartments filled with hydrogel.

The compartments of the bottom and cover are elongate and extend in the same direction. When the hydrogel is saturated with water the hydrogel expands, whereby the compartments become more or less cylindrical. The compartments of the bottom and of the cover here come to lie closer against each other. This is an advantageous form for cooling insulin syringes because the insulin syringes can be placed in the cavities between the cylindrical compartments. The insulin syringes can in this way be properly cooled.

Instead of injecting insulin with an insulin syringe at a number of times during the day, it is also possible to wear an insulin pump. The insulin pump administers a small quantity of insulin to the patient over the course of the day. The insulin pump is normally worn on a trouser belt. The insulin in the pump will as a result warm up quickly.

It is now found that administering warmed insulin can result in nausea. This is because, among other reasons, air bubbles can occur as the insulin becomes warmer. It is therefore desirable to cool the insulin in the pump.

When the cooled prior art pouch is used, little or no further space will be available in the pouch for the insulin pump when the hydrogel is fully saturated, whereby the cover of the pouch remains open. Much of the cold will hereby dissipate to the environment.

In the case the internal space of the pouch is enlarged so that the cover can close, play will then occur in the pouch after water has evaporated from the hydrogel, whereby the insulin pump comes to lie loosely in the pouch, lies less well against the walls and cooling is reduced.

It is now an object of the invention to reduce or even obviate the above stated drawbacks.

This object is achieved according to the invention with a pouch according to the preamble, characterized in that at least two opposite wall parts of the peripheral wall are provided with hydrogel-filled compartments and wherein the wall parts are water-permeable for the purpose of bringing the hydrogel into contact with water.

Because in the pouch according to the invention at least two wall parts of the peripheral wall are provided with compartments with hydrogel, the insulin pump can be pushed between these two walls without the wall parts moving apart from each other. The wall parts are after all connected to each other to form a form-closed peripheral wall. The insulin pump can thus even be pressed under some tension between the wall parts.

Because the compartments with hydrogel are arranged in the peripheral wall, it becomes possible to press the compartments with more tension against the insulin pump so that, even after evaporation of water, the compartments still lie properly against the walls of the insulin pump. The insulin in the insulin pump will hereby remain cooled for a sustained period, whereby nausea and air bubble formation are prevented.

In a preferred embodiment of the pouch according to the invention the wall parts provided with hydrogel-filled compartments comprise a first water-permeable layer and a second layer, wherein the first layer and the second layer are attached to each other along the periphery as well as being attached to each other along attachment lines, which attachment lines extend between the edges of the layers in order to form compartments.

By providing two layers, of which at least one layer is water-permeable, a pouch can be manufactured in a simple manner. The layers can for instance be attached to each other along the periphery by stitching and the attachment lines can also be provided by stitches. Other attaching methods, such as ultrasonic welding, are otherwise also possible.

By making at least the first layer water-permeable the hydrogel can become saturated by for instance immersing the pouch in water, and during use the water can evaporate from the hydrogel via the water-permeable layer. The second layer can also be water-permeable or, conversely, watertight in order to protect a part lying therebehind, such as an insulin pump, from moisture.

The first and second layers can for instance be of cotton or a breathing material. The weight of the layer used is here preferably 140 g/m² or more in order to ensure that the pouch is sufficiently firm and can withstand regular removal and re-placement of the insulin pump.

The compartments formed between the layers are preferably elongate and the longitudinal axes of the compartments extend parallel to the bottom wall.

In a highly preferred embodiment of the pouch according to the invention the distance over the surface and between the longitudinal edges of a compartment is less than 2 centimetres, preferably less than or equal to 1.25 centimetre.

Keeping the distance over the surface and between the longitudinal edges of a compartment to less than 2 centimetres ensures that the compartment with saturated hydrogel does not become larger than a circular periphery of twice 2 centimetres. This ensures that the change in thickness of the wall part remains limited and an insulin pump can more easily be held in direct contact with the compartments for a long period.

It is highly preferred to keep the distance to less than 1.25 centimetre. It is found here that sufficient cooling capacity is retained while the thickness of the pouch remains limited.

Each compartment is preferably filled with a maximum of 3.0 g hydrogel, preferably a maximum of 2.0 g hydrogel. This also ensures that the compartments do not swell too much, whereby there would be insufficient space for an insulin pump in the pouch, or that there is too much space in the pouch when a large part of the water has evaporated from the hydrogel.

In another embodiment of the pouch according to the invention the bottom wall is provided with at least one compartment filled with hydrogel. The cooling capacity of the pouch is increased by also providing the bottom of the pouch with a compartment. The insulin pump will moreover sink automatically against the bottom because of the gravitational force while being worn on the belt of the user, and so remain in good heat-exchanging contact with the compartment in the bottom.

It is further also possible according to the invention to provide all walls of the pouch with compartments.

In a further preferred embodiment of the pouch according to the invention the wall parts extending between the at least two opposite wall parts provided with hydrogel-filled compartments are provided along the insertion opening with an elastic strip.

The elastic strip ensures that the wall parts of the peripheral wall with the compartments can be pressed apart so that space is obtained for the insulin pump, and that the wall parts with the compartments remain pressed against the insulin pump by the resilience of the elastic strips, even when a large part of the water has evaporated from the hydrogel.

In yet another embodiment of the pouch according to the invention a removable protective layer, such as a layer of leather or plastic, is arranged against the outer side of at least the bottom wall and against the wall parts provided with compartments.

The wall parts with the hydrogel-filled compartments are water-permeable in order to enable wetting of the hydrogel and to enable evaporation of the moisture from the hydrogel. A direct contact of the moist wall parts with the skin of a user can be uncomfortable. Direct contact is now avoided by arranging a protective layer. In order to allow sufficient evaporation of the moisture in the hydrogel the protective layer can be perforated or provided with small apertures. In such a variant there will also be no direct contact between the user and the moist wall parts.

In yet another embodiment of the pouch according to the invention the protective layer forms the wall parts of the peripheral wall which are not provided with compartments such that the protective layer forms a bag, and wherein the bottom wall and the wall parts provided with compartments form a mat removable from the bag.

When a protective layer is used, a bag can be formed easily therefrom in which the bottom and wall parts with hydrogel-filled compartments are arranged releasably as a mat. The bag can thus be used to store the insulin pump, even when the hydrogel-filled compartments have been removed, for instance in order to be re-wetted.

In a further embodiment of the pouch according to the invention an elastic part is arranged in at least one of the wall parts of the peripheral wall which are not provided with compartments.

The elastic wall part can ensure that the bag encloses the insulin pump properly even when the mat with compartments has been taken out of the bag.

In yet another embodiment of the pouch according to the invention the inner side of at least the bottom wall and the wall parts provided with compartments is water-impermeable.

Making the inner side of the pouch water-impermeable prevents an insulin pump arranged in the pouch coming into direct contact with moisture. Making the inner side water-impermeable does away with the necessity of giving the insulin pump itself a watertight form or of arranging a protective casing around the insulin pump.

The invention further comprises a combination of a pouch according to the invention and a protective casing, which protective casing is arranged in the pouch for removal via the insertion opening and wherein the protective casing has a closable insertion opening for insertion of an insulin pump.

The protective casing is preferably at least water-repellent.

The protective casing can be specifically adapted to the protection required for the insulin pump, for instance protection from moisture, while the pouch according to the invention can thus be specifically adapted in order to generate a maximum cooling capacity.

A further bag can also be provided into which the pouch and optional protective casing can be placed. It is thus possible to give the bag an exclusive appearance, to design the pouch specially for a maximum cooling capacity and to embody the protective casing for optimum protection of the insulin pump. The bag can for instance be made from a desired type of leather, such as calfskin or oxhide, or from a suitable plastic.

A further embodiment of the combination according to the invention further comprises an insulin pump, which insulin pump has a substantially block-shaped housing and wherein the insulin pump is arranged between the at least two opposite wall parts of the peripheral wall of the pouch.

These and other features of the invention are further elucidated with reference to the accompanying drawings.

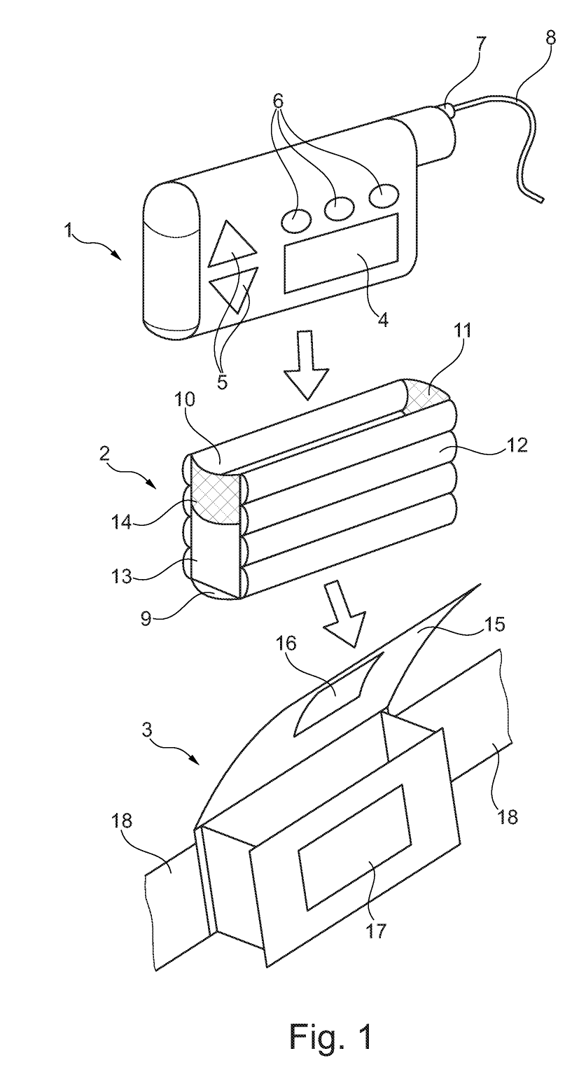

FIG. 1 shows an embodiment of the combination according to the invention with exploded parts.

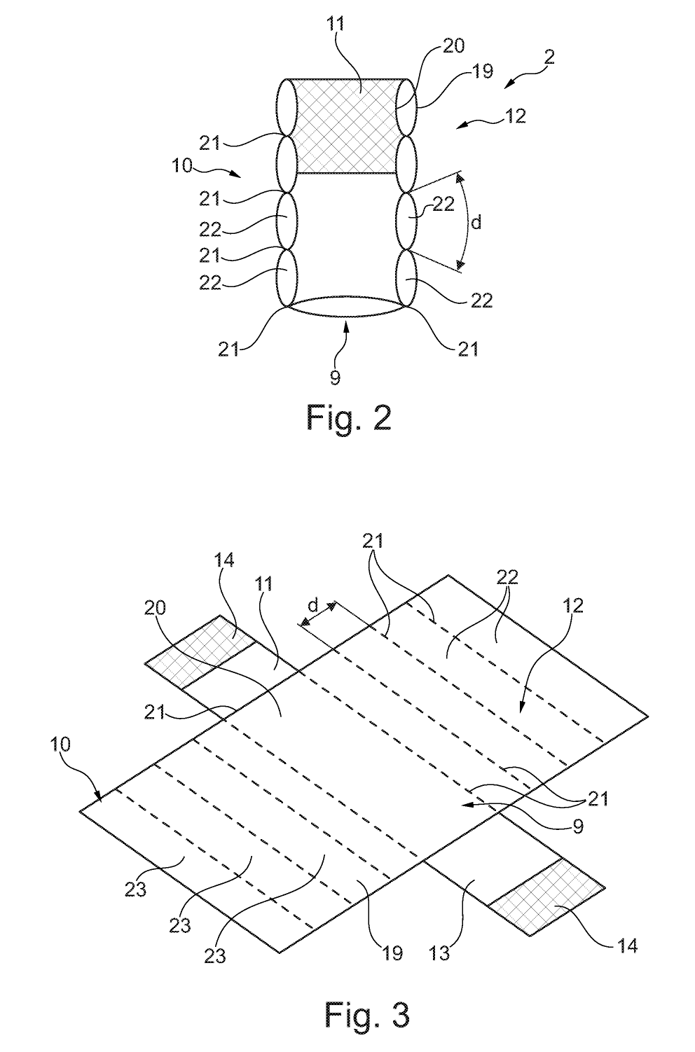

FIG. 2 shows a cross-sectional view of the pouch according to the invention.

FIG. 3 shows a blank of the pouch according to FIG. 2.

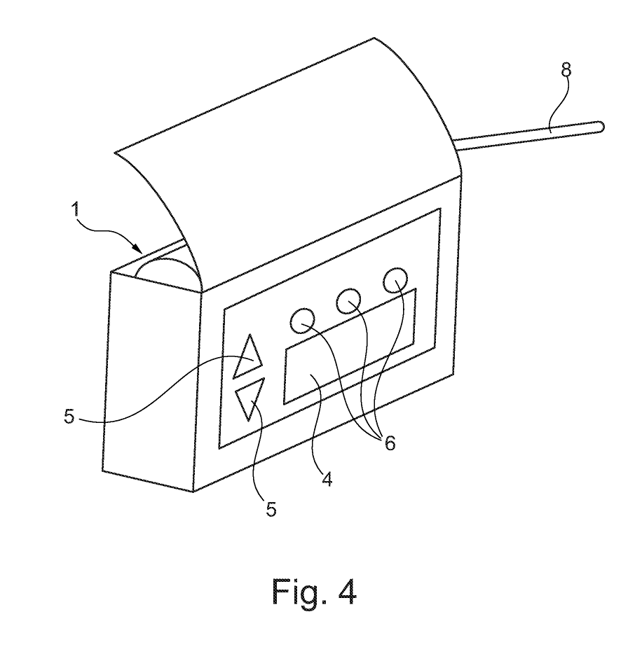

FIG. 4 shows a protective casing according to the invention.

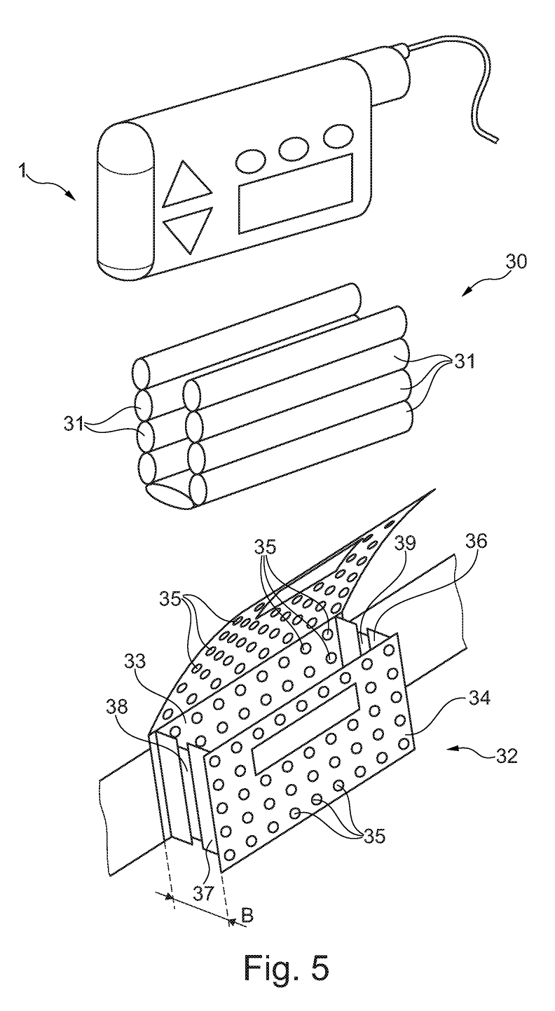

FIG. 5 shows a variant of the combination according to FIG. 1.

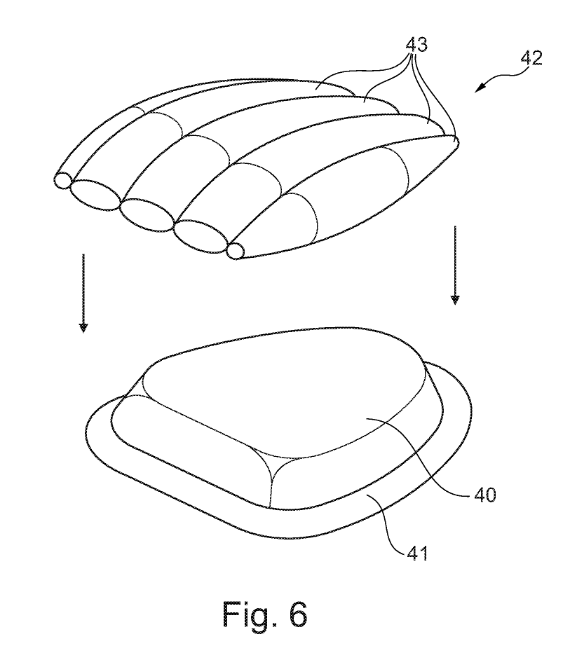

FIG. 6 shows a third embodiment of the combination according to the invention.

FIG. 1 is a perspective view of a combination 1 according to the invention. The combination has an insulin pump 1, a pouch 2 and a hip bag 3.

Insulin pump 1 has a substantially block-shaped housing in which a screen 4 and several control buttons 5, 6 are arranged. Further provided on the block-shaped housing is a hose connection 7 for a hose 8 with which insulin pump 1 can administer insulin to a patient.

Insulin pump 1 is placed during use in a pouch 2. This pouch 2 has a bottom 9 with a peripheral wall with wall parts 10, 11, 12, 13. The long wall parts 10, 12 and bottom 9 are provided with compartments (see FIG. 2). The upper edge of wall parts 10, 11, 12, 13 form an insertion opening through which the insulin pump 1 can be placed into pouch 2.

In this embodiment the short wall parts 11, 13 take a single-walled form and are provided with an elastic strip 14 with which the long wall parts 10, 12 can be pressed against the housing of insulin pump 1.

Pouch 2 together with the inserted insulin pump 1 is then placed in a hip bag 3. This hip bag 3 has a flap 15 which is provided with Velcro tape 16, 17 with which hip bag 3 can be closed.

Further provided on hip bag 3 is a belt 18 with which bag 3 can be easily arranged around the waist of a patient.

FIG. 2 shows a cross-section of pouch 2. Wall parts 10 and 12 as well as bottom 9 have a first layer 19 and a second layer 20 which are connected to each other by means of stitching 21 and whereby compartments 22 are formed (see also FIG. 3).

A quantity of hydrogel 23 is arranged in compartments 22. Owing to the water-permeability of layers 19 and 20 this hydrogel 23 can absorb water which subsequently evaporates again so that cooling takes place. Insulin pump 1 placed in pouch 2 can hereby be cooled.

It will further be apparent from FIG. 3 that a further two wall parts 11, 13 are arranged on the two layers 19, 20 forming the wall parts with compartments 22. On the upper side of these wall parts 11, 13 elastic strips 14 are provided close to the insertion opening of pouch 2 in order to press wall parts 10, 12 with compartments 22 against insulin pump 1 and thus keep it cooled in that the hydrogel 23 evaporates water.

Hydrogel 23 can be provided as granular material or as beads. A possible active component of the hydrogel are acid sodium compounds with cross-linked polypropylene).

FIG. 4 shows a protective casing 24 which can be arranged round insulin pump 1. Protective casing 24 has a cover 25 with which casing 24 can be closed and with which insulin pump 1 can be protected from moisture from pouch 2.

FIG. 5 shows a variant of the combination according to FIG. 1 with exploded parts. In this variant a mat 30 with compartments 31 extending in longitudinal direction is wrapped around insulin pump 1. Mat 30 thus forms the bottom and two wall parts of the peripheral wall of a pouch according to the invention.

This mat 30 is then placed together with insulin pump 1 in a bag 32. This bag 32 is formed from a layer of leather or plastic which provides protection for mat 30 and insulin pump 1. Bag 32 has two longitudinal walls 33, 34 which are arranged against compartments 31 of mat 30. Longitudinal walls 33, 34 are provided with perforations 35 so that the moisture in the hydrogel in compartments 31 can evaporate easily via perforations 35.

Bag 32 further has side walls 36, 37 in which an elastic part 38, 39 is arranged. This elastic part 38, 39 ensures that the width B of bag 32 can vary so that bag 32 can enclose insulin pump 1 properly with and without mat 30.

FIG. 6 shows a third embodiment of the combination according to the invention. Shown herein is an insulin pump 40 which can be arranged on the skin of a user using a plaster 41. A pouch 42 is then placed round this insulin pump. This pouch 42 is provided over the whole surface with compartments 43 filled with a hydrogel.

If desired, a further protective layer (not shown) can be arranged over pouch 42 in the same way as in the embodiment shown in FIG. 1 or 5.

1. A pouch for cooling an insulin pump, comprising:
a bottom wall; and
a standing peripheral wall along the bottom wall for enclosing an insulin pump and wherein an upper edge of the peripheral wall forms an insertion opening for inserting the insulin pump;
wherein at least two opposite wall parts of the peripheral wall are provided with hydrogel-filled compartments and wherein the wall parts are water-permeable for the purpose of bringing the hydrogel into contact with water.

2. The pouch as claimed in claim 1, wherein the wall parts provided with hydrogel-filled compartments comprise a first water-permeable layer and a second layer, wherein the first layer and the second layer are attached to each other along the periphery as well as being attached to each other along attachment lines, which attachment lines extend between the edges of the layers in order to form compartments.

3. The pouch as claimed in claim 2, wherein the compartments formed between the layers are elongate and the longitudinal axes of the compartments extend parallel to the bottom wall.

4. The pouch as claimed in claim 3, wherein the distance over the surface and between the longitudinal edges of a compartment is less than 2 centimetres.

5. The pouch as claimed in claim 3, wherein each compartment is filled with a maximum of 3.0 g hydrogel.

6. The pouch as claimed in claim 1, wherein the bottom wall is provided with at least one compartment filled with hydrogel.

7. The pouch as claimed in claim 1, wherein the wall parts extending between the at least two opposite wall parts provided with hydrogel-filled compartments are provided along the insertion opening with an elastic strip.

8. The pouch as claimed in claim 1 , wherein a removable protective layer is arranged against an outer side of at least the bottom wall and against the wall parts provided with compartments.

9. The pouch as claimed in claim 8, wherein the protective layer forms the wall parts of the peripheral wall which are not provided with compartments such that the protective layer forms a bag, and wherein the bottom wall and the wall parts provided with compartments form a mat removable from the bag.

10. The pouch as claimed in claim 9, wherein an elastic part is arranged in at least one of the wall parts of the peripheral wall which are not provided with compartments.

11. The pouch as claimed in claim 1, wherein an inner side of at least the bottom wall and the wall parts provided with compartments is water-impermeable.

12. A combination of a pouch as claimed in claim 1 and a protective casing, which protective casing is arranged in the pouch for removal via the insertion opening and wherein the protective casing has a closable insertion opening for insertion of an insulin pump.

13. The combination as claimed in claim 12, wherein the protective casing is at least water-repellent.

14. The combination as claimed in claim 12, further comprising an insulin pump, which insulin pump has a substantially block-shaped housing and wherein the insulin pump is arranged between the at least two opposite wall parts of the peripheral wall of the pouch.

15. The pouch as claimed in claim 4, wherein each compartment is filled with a maximum of 3.0 g hydrogel.

16. The pouch as claimed in claim 5, wherein each compartment is filled with a maximum of 2.0 g hydrogel.

17. The pouch as claimed in claim 4, wherein the distance over the surface and between the longitudinal edges of a compartment is less than or equal to 1.25 centimetres.

18. The pouch as claimed in claim 8, wherein the protective layer comprises leather or plastic.

19. The pouch as claimed in claim 2, wherein the bottom wall is provided with at least one compartment filled with hydrogel.

20. The combination as claimed in claim 13, further comprising an insulin pump, which insulin pump has a substantially block-shaped housing and wherein the insulin pump is arranged between the at least two opposite wall parts of the peripheral wall of the pouch.
